(19) United States
(12) Patent Application Publication
(10) Pub. No.: US 2013/0158492 A1
(43) Pub. Date: Jun. 20, 2013

(54) Absorbent Article With Moisture Indicator

(71) Applicants: Xuedong Song, Alpharetta, GA (US); FuLiang Cheng, Taoyuan (TW); Karen Meloy Goeders, Atlanta, GA (US); Yong Li, Kingsport, TN (US)

(72) Inventors: Xuedong Song, Alpharetta, GA (US); FuLiang Cheng, Taoyuan (TW); Karen Meloy Goeders, Atlanta, GA (US); Yong Li, Kingsport, TN (US)

(21) Appl. No.: 13/332,109

(22) Filed: Dec. 20, 2011

(51) Int. Cl. A61L 15/56; G01N 21/78

(52) U.S. Cl. 604/361; 436/39

(57) ABSTRACT

A personal absorbent article (e.g., feminine care pad, sanitary napkin, diaper, incontinence pad etc.) has a color change indicator that employs a synergistic combination an acid component and a basic component for signaling to a user when the absorbent article may foster bacterial growth due to moisture absorption. After a certain period of time, the user can visually inspect the color of the color change indicator to obtain an indication of when it is time to replace or remove the article.

### BACKGROUND OF THE INVENTION

The present disclosure relates to personal care absorbent articles. More specifically, the disclosure relates to indicators that may be used in absorbent articles to provide the wearer with a signal which suggests that the absorbent article needs to be replaced.

A woman's vagina is naturally colonized by a variety of bacteria, yeast, and microorganisms. For example, a normal vagina generally contains more than about 104 lactobacilli per milliliter of vaginal material. Under normal conditions, the vaginal flora provides a mildly acidic environment that helps guard against the invasion of pathogenic microbes (e.g., Gardnerella vaginalis, Candida albicans, etc.). Unfortunately, this vaginal balance may be easily upset by a variety of factors that ultimately lead to vaginal infection.

One such factor is that women may not timely change an absorbent article if it is only moistened by sweat. For example, in warmer climates a woman may experience a high degree of sweating near the vaginal region. Absorbent articles, by necessity, are capable of storing moisture emanating from sweat, other bodily fluids or the environment and liquids for a long period of time. An increase in the absorbent article's moisture content may, over an extended period of time, stimulate the growth of pathogenic microorganisms that could potentially lead to infection and discomfort. It is not necessary for a liquid insult to occur for there to be moisture in an absorbent article, the presence of sweat is all that is needed.

Not only are women at risk for infection by prolonged use of absorbent articles moistened from sweat or the like, children and incontinent adults can experience the same issue. Diapers, training pants, incontinence pads and incontinence pants are also worn in hot climates where sweating is apt to occur. Not only might the moisture increase create a condition bacteria can thrive, but skin irritation may occur as well. Diapers have employed chemically reactive wetness indicators for some time. Generally, wetness indicators exhibit a color change when in contact with a significant amount of liquid. However, such an indicator is not useful when it is only water vapor or insignificant amounts of liquid that moisten the absorbent article. In such a case, moisture derived from sweat may not activate the wetness indicator, potentially leaving a wearer exposed to a moist absorbent article for an extended period of time.

As such, a need currently exists for an absorbent article that is well suited for conditions where the absorbent article contains moisture from the environment and the body, and that can provide a signal to suggest to the user that the absorbent article needs to be changed.

### SUMMARY OF THE INVENTION

In accordance with one embodiment of the present disclosure, provided is a color change indicator for a substrate, wherein the color change indicator includes a pH indicator composition, an acid component and a base component. The acid component and base component are physically separated and configured to chemically react with one another in the presence of an effective amount of water.

In one aspect, an absorbent article has an absorbent core positioned between the baffle and the topsheet. The color change indicator is disposed on the baffle and/or the absorbent core and/or the topsheet of the absorbent article.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figure in which:

FIG. 1 is a top view of one embodiment of a feminine care absorbent article of the present invention;

FIG. 2 is a plan view of another embodiment of a feminine care absorbent article of the present invention;

FIG. 3 is a front perspective view of one embodiment of a pant of the present invention;

FIG. 4 is a plan view of the pant depicted in FIG. 3

FIG. 5 is a plan view of one embodiment of a color-change indicator stick of the present invention; and

FIG. 6 is a cross-sectional view of the color-change indicator stick shown in FIG. 5, taken at line 6-6.

Repeat use of references characters in the present specification and drawing is intended to represent same or analogous features or elements of the invention.

### DETAILED DESCRIPTION

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

The term “humidity” as used herein refers to water vapor from the environment to which an absorbent article is exposed.

The term “moisture” as used herein refers to an aqueous liquid that is not able flow as a liquid within an absorbent product. Moisture is generally water vapor or condensed water vapor that moves into and through an absorbent article by wicking. The water vapor or condensed water vapor may emanate from the environment and/or bodily exudates such as sweat.

Generally speaking, the present invention is directed to an absorbent article (e.g., feminine care pad, sanitary napkin, pant, etc.) that employs a color change indicator sensitive to a combination of moisture, temperature and exposure time. A pre-set condition of moisture, temperature and time is used to trigger color change(s) of the indicators to suggest to wearers or caretakers (collectively referred to as “users”) that the condition of the absorbent article is not optimal for wear.

In one aspect of the disclosure, the color of the indicator remains unchanged for a relatively long period of time (e.g., >6 hours) when it is exposed to a combination of a certain temperature (e.g., 35 C) and relatively low humidity (e.g., <40%). In another aspect, the color of the indicator changes when exposed for a relatively short time (e.g., <2 hours) when exposed to a combination of relatively high temperature (e.g., >35 C) and humidity (>70%). In yet another aspect, the color of the indicator remains unchanged for a moderate period of time (e.g., from 2 to 6 hours) when exposed to a combination of relatively high humidity (e.g., >70%) and relatively low temperature (e.g., <30 C) or a combination of a relatively low humidity (e.g., <40%) and high temperature (e.g., >35 C), or a combination of moderate temperature (e.g., from 30 to 35 C) and moderate humidity (from 40% to 70%).

The present disclosure is directed to a color-change indicator that exhibits a color change when exposed to moisture. In one aspect, the color-change indicator includes an acid component and a base component that remain segregated until there is an effective amount of moisture present to cause migration of one or both components. Specific aspects of such embodiments are described herein.

The color change indicator may be used on various types of absorbent articles used to absorb bodily exudates. One absorbent article of the present disclosure is a feminine care article that includes at least one generally liquid-impermeable layer (e.g., outer cover or baffle), at least one liquid-permeable layer (e.g., topsheet, intake layer, transfer delay layer, etc.), and an absorbent core positioned between the liquid-impermeable layer and liquid-permeable layer. The color change indicator of the present disclosure may generally be disposed in fluid communication with any of these components in a variety of different orientations and configurations. Nevertheless, in most embodiments, it is desired that the color change indicator is applied to a portion of the article where it can be easily seen by the user, such as a liquid-impermeable layer and/or the liquid permeable layer.

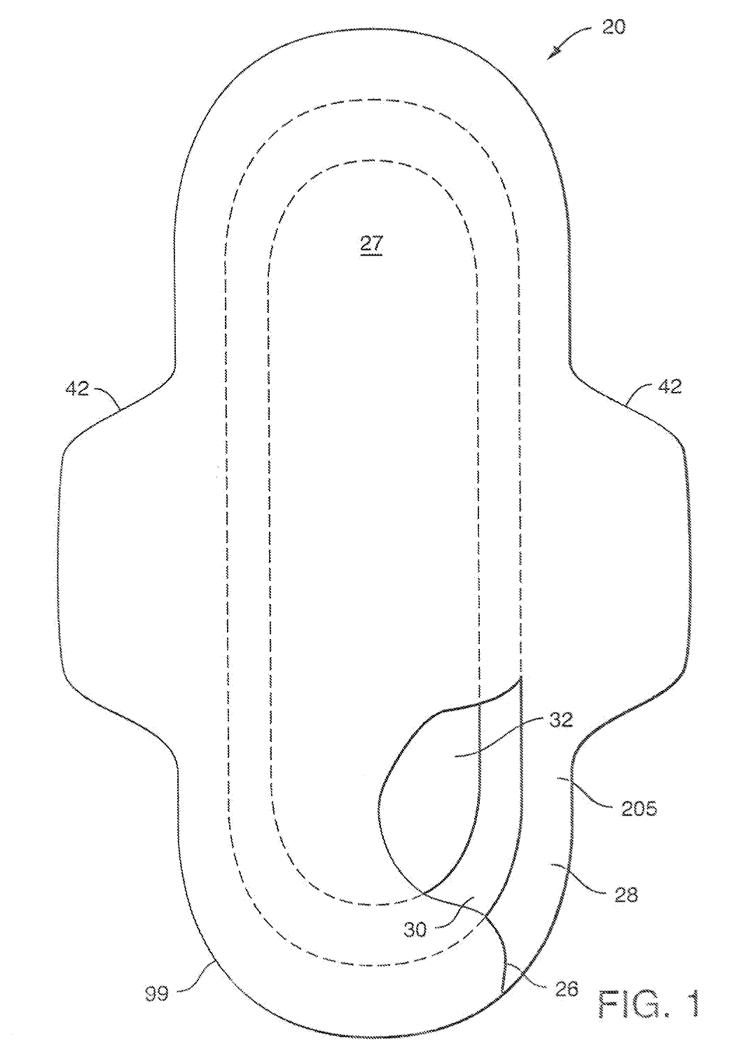

Referring to FIG. 1, one particular embodiment of a feminine care absorbent article 20 will now be described in more detail. More particularly, the article 20 includes a topsheet 26, a baffle 28, and an absorbent core 30 positioned between the topsheet 26 and the baffle 28. The topsheet 26 defines a body-facing surface 27 of the absorbent article 20. The absorbent core 30 is positioned inwardly from the outer periphery of the absorbent article 20 and includes a body-facing side positioned adjacent the topsheet 26 and a garment-facing surface positioned adjacent the baffle 28. Typically, the topsheet 26 and the baffle 28 are joined by adhesive bonding, ultrasonic bonding, or any other suitable joining method known in the art, the sealed edges defining an overall sealed peripheral edge 99 of the article 20. The article 20 may take on various geometries but will generally have opposite lateral sides and longitudinal ends.

The topsheet 26 is generally designed to contact the body of the user and is liquid-permeable. The liquid permeable topsheet 26 has an outwardly facing surface that may contact the body of the user and receive aqueous fluids from the body. The topsheet 26 is provided for comfort and conformability and functions to direct bodily exudates away from the body, through the topsheet 26 and toward the absorbent core 30. The topsheet 26 retains little or no liquid in its structure so that it provides a relatively comfortable and non-irritating surface next to the tissues within the vestibule of a female user. The topsheet 26 may be constructed of any woven or nonwoven material that is easily penetrated by bodily exudates contacting the surface of the baffle. Examples of suitable materials include rayon, bonded carded webs of polyester, polypropylene, polyethylene, nylon, or other heat-bondable fibers, polyolefins, such as copolymers of polypropylene and polyethylene, linear low-density polyethylene, and aliphatic esters such as polylactic acid. Finely perforated film webs and net material can also be used. A specific example of a suitable topsheet material is a bonded carded web made of polypropylene and polyethylene such as that used as topsheet stock for KOTEX® pantiliners and obtainable from Sandler Corporation (Germany). U.S. Pat. Nos. 4,801,494 to Datta, et al. and 4,908,026 to Sukiennik, et al. teach various other topsheet materials that may be used in the present disclosure. The topsheet 26 may also contain a plurality of apertures (not shown) formed therethrough to permit body fluid to pass more readily into the absorbent core 30. The apertures may be randomly or uniformly arranged throughout the topsheet 26, or they may be located only in the narrow longitudinal band or strip arranged along the longitudinal axis of the absorbent article 20. The apertures permit rapid penetration of body fluid down into the absorbent core 30. The size, shape, diameter and number of apertures may be varied to suit one's particular needs.

The baffle 28 is generally liquid-impermeable and designed to face the inner surface, i.e., the crotch portion of an undergarment (not shown). The baffle 28 may permit a passage of air or vapor out of the absorbent article 20, while still blocking the passage of liquids. Any liquid-impermeable material may generally be utilized to form the baffle 28. For example, one suitable material that may be utilized is a microporous polymeric film, such as polyethylene or polypropylene. In particular embodiments, a polyethylene film is utilized that has a thickness in the range of about 0.2 mils to about 5.0 mils, and particularly between about 0.5 to about 3.0 mils. A specific example of a baffle material is a polyethylene film such as that used in KOTEX® pantiliners and obtainable from Pliant Corporation, Schaumburg, Ill., USA.

As indicated above, an absorbent core 30 is positioned between the topsheet 26 and the baffle 28 that provides capacity to absorb and retain bodily exudates. The absorbent core 30 may be formed from a variety of different materials and contain any number of desired layers. For example, the core 30 typically includes one or more layers of an absorbent web material of cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In a particular embodiment, the absorbent web material includes a matrix of cellulosic fluff, and may also include superabsorbent material. The cellulosic fluff may comprise a blend of wood pulp fluff. One preferred type of fluff is identified with the trade designation NB 416, available from Weyerhaeuser Corp., and is a bleached, highly absorbent wood pulp containing primarily soft wood fibers. The absorbent materials may be formed into a web structure by employing various conventional methods and techniques. For example, the absorbent web may be formed with a dry-forming technique, an air forming technique, a wet-forming technique, a foam-forming technique, or the like, as well as combinations thereof. A coform nonwoven material may also be employed. Methods and apparatus for carrying out such techniques are well known in the art.

The topsheet 26 may be maintained in secured relation with the absorbent core 30 by bonding all or a portion of the adjacent surfaces to one another. A variety of bonding mechanisms known to one of skill in the art may be utilized to achieve any such secured relation. Examples of such mechanisms include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent. The topsheet 26 typically extends over the upper, bodyside surface of the absorbent core 30, but can alternatively extend around the article to partially or entirely, surround or enclose the absorbent core. Alternatively, the topsheet 26 and the baffle 28 can have peripheral margins that extend outwardly beyond the terminal, peripheral edges of the absorbent core 30, and the extending margins may be joined together to partially or entirely, surround or enclose the absorbent core.

Although not required, the absorbent article 20 may also contain other additional layers as is known in the art. In FIG. 1, for example, a liquid-permeable intake layer 32 is positioned vertically between the topsheet 26 and the absorbent core 30. The intake layer 32 may be made of a material that is capable of rapidly transferring, in the z-direction, body fluid that is delivered to the topsheet 26. The intake layer 32 may generally have any shape and/or size desired. In one embodiment, the intake layer 32 has a rectangular shape, with a length equal to or less than the overall length of the absorbent article 20, and a width less than the width of the absorbent article 20. For example, a length of between about 150 mm to about 300 mm and a width of between about 10 mm to about 60 mm may be utilized. Any of a variety of different materials may be used for the intake layer 32 to accomplish the above-mentioned functions. The material may be synthetic, cellulosic, or a combination of synthetic and cellulosic materials. For example, airlaid cellulosic tissues may be suitable for use in the intake layer 32. The airlaid cellulosic tissue may have a basis weight ranging from about 10 grams per square meter (gsm) to, about 300 gsm, and in some embodiments, between about 40 gsm to about 150 gsm. The airlaid tissue may be formed from hardwood and/or softwood fibers. The airlaid tissue has a fine pore structure and provides an excellent wicking capacity, especially for menses.

The absorbent article 20 may also contain a transfer delay layer (not shown) positioned between the intake layer 32 and the absorbent core 30. The transfer delay layer may contain a material that is substantially hydrophobic, such as a nonwoven web composed of polypropylene, polyethylene, polyester, etc. One example of a material suitable for the transfer delay layer is a spunbond web composed of polypropylene, multi-lobal fibers. Further examples of suitable transfer delay layer materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay layer 36 are described in U.S. Pat. Nos. 4,798,603 to Meyer, et al. and 5,248,309 to Serbiak, et al. To adjust performance, the transfer delay layer may also be treated with a selected amount of surfactant to increase its initial wettability. The transfer delay layer typically has a basis weight less than that of the other absorbent members. For example, the basis weight of the transfer delay layer 36 is typically less than about 250 grams per square meter (gsm), and in some embodiments, between about 40 gsm to about 200 gsm.

The absorbent article 20 may also include laterally extending wing portions 42 that may be integrally connected to side regions along the intermediate portion of the article. For example, the wing portions 42 may be separately provided members that are subsequently attached or otherwise operatively joined to the intermediate portion of the article. In other configurations, the wing portions may be unitarily formed with one or more components of the article. As representatively shown in FIG. 1, for example, either or both wing portions 42 may be formed from a corresponding, operative extension of the material employed to form the topsheet 26. Alternatively, either or both wing portions 42 may be formed from a corresponding, operative extension of the material employed to form the baffle 28, or formed from a corresponding, operative combination of the topsheet and baffle materials.

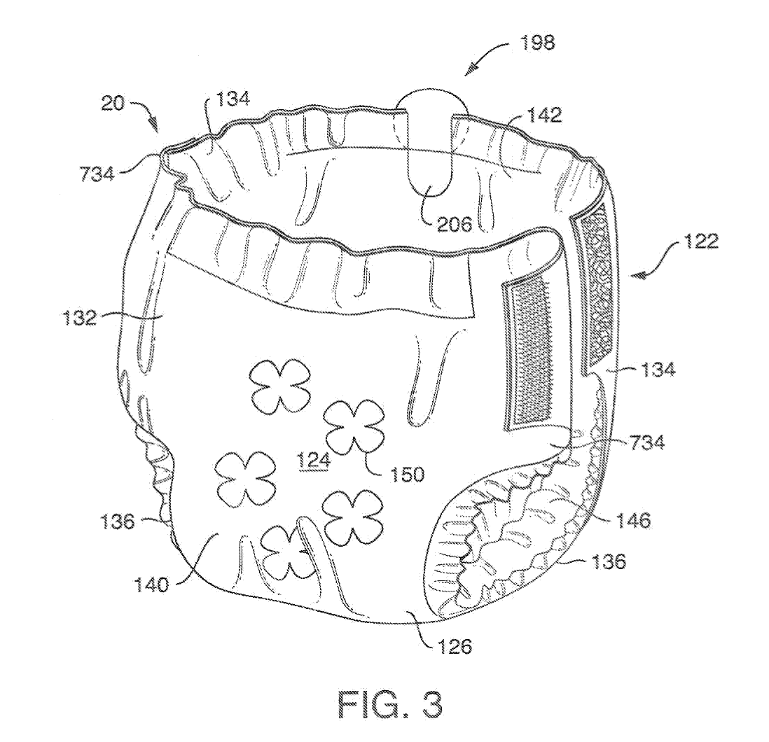

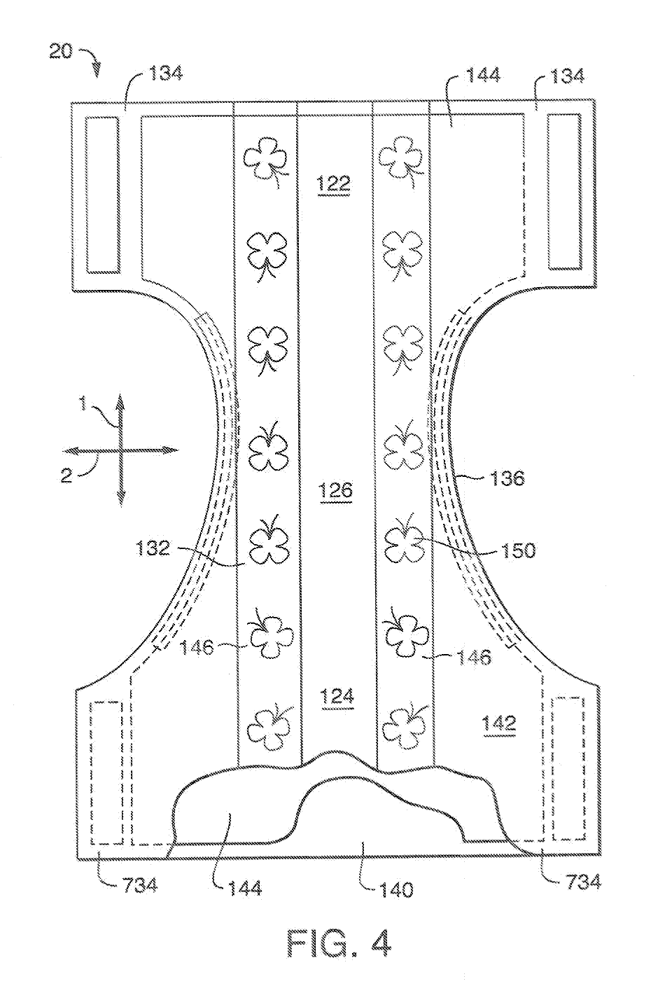

The color change indicator is also useful in disposable pant- or diaper-type absorbent articles. Such an absorbent article of the present disclosure generally can have an absorbent core, and can optionally include a topsheet and/or a baffle, where the absorbent core can be disposed between the topsheet and the baffle. To gain a better understanding of the present disclosure, attention is directed to FIGS. 3 and 4 for exemplary purposes showing a training pant with a color-change indicatory of the present disclosure.

Various materials and methods for constructing training pants are disclosed in U.S. Pat. No. 6,761,711 to Fletcher et al.; U.S. Pat. No. 4,940,464 to Van Gompel et al.; U.S. Pat. No. 5,766,389 to Brandon et al., and U.S. Pat. No. 6,645,190 to Olson et al., each of which is incorporated herein by reference in a manner that is consistent herewith.

FIG. 3 illustrates a training pant 20 in a partially fastened condition, and FIG. 4 illustrates a training pant 20 in an opened and unfolded state. The training pant 20 defines a longitudinal direction 1 that extends from the front of the training pant when worn to the back of the training pant. Perpendicular to the longitudinal direction 1 is a lateral direction 2.

The training pant 20 defines a front region 122, a back region 124, and a crotch region 126 extending longitudinally between and interconnecting the front and back regions. The pant 20 also defines an inner surface (i.e., body-facing surface) adapted in use (e.g., positioned relative to the other components of the pant) to be disposed toward the wearer, and an outer surface (i.e., garment-facing surface) opposite the inner surface. The training pant 20 has a pair of laterally opposite side edges and a pair of longitudinally opposite waist edges.

The illustrated pant 20 may include a chassis 132, a pair of laterally opposite front side panels 134 extending laterally outward at the front region 122 and a pair of laterally opposite back side panels 734 extending laterally outward at the back region 24.

The chassis 132 includes a baffle 140 and a topsheet 142 that may be joined to the baffle 140 in a superimposed relation therewith by adhesives, ultrasonic bonds, thermal bonds or other conventional techniques. The chassis 132 may further include an absorbent core 144 such as shown in FIG. 4 disposed between the baffle 140 and the topsheet 142 for absorbing fluid body exudates exuded by the wearer, and may further include a pair of containment flaps 146 secured to the topsheet 142 or the absorbent core 144 for inhibiting the lateral flow of body exudates.

The baffle 140, the topsheet 142 and the absorbent core 144 may be made from many different materials known to those skilled in the art. The baffle 140 may be constructed of a nonwoven material. The baffle 140, may be a single layer of a fluid impermeable material, or alternatively may be a multi-layered laminate structure in which at least one of the layers is fluid impermeable. The baffle 140 may allow water vapor to pass therethrough.

Examples of suitable baffle 140 materials are spunbond-meltblown fabrics, spunbond-meltblown-spunbond fabrics, spunbond fabrics, or laminates of such fabrics with films, or other nonwoven webs; elastomeric materials that may include cast or blown films, meltblown fabrics or spunbond fabrics composed of polyethylene, polypropylene, or polyolefin elastomers, as well as combinations thereof. The baffle 140 may include materials that have elastomeric properties through a mechanical process, printing process, heating process or chemical treatment. For example, such materials may be apertured, creped, neck-stretched, heat activated, embossed, and micro-strained, and may be in the form of films, webs, and laminates.

One example of a suitable material for a biaxially stretchable baffle 140 is a breathable elastic film/nonwoven laminate, such as described in U.S. Pat. No. 5,883,028, to Morman et al., incorporated herein by reference in a manner that is consistent herewith. Examples of materials having two-way stretchability and retractability are disclosed in U.S. Pat. Nos. 5,116,662 to Morman and 5,114,781 to Morman, each of which is incorporated herein by reference in a manner that is consistent herewith.

The topsheet 142 is suitably compliant, soft-feeling and non-irritating to the wearer's skin. The topsheet 142 is also sufficiently liquid permeable to permit liquid body exudates to readily penetrate through its thickness to the absorbent core 144. A suitable topsheet 142 may be manufactured from a wide selection of web materials, such as porous foams, reticulated foams, apertured plastic films, woven and non-woven webs, or a combination of any such materials. For example, the topsheet 142 may include a meltblown web, a spunbonded web, or a bonded-carded-web composed of natural fibers, synthetic fibers or combinations thereof. The topsheet 142 may be composed of a substantially hydrophobic material, and the hydrophobic material may optionally be treated with a surfactant or otherwise processed to impart a desired level of wettability and hydrophilicity.

The topsheet 142 may also be extensible and/or elastomerically extensible. Suitable elastomeric materials for construction of the topsheet 142 can include elastic strands, LYCRA elastics, cast or blown elastic films, nonwoven elastic webs, meltblown or spunbond elastomeric fibrous webs, as well as combinations thereof. Examples of suitable elastomeric materials include KRATON elastomers, HYTREL elastomers, ESTANE elastomeric polyurethanes (available from Noveon, a business having offices located in Cleveland, Ohio U.S.A.), or PEBAX elastomers. The topsheet 142 can also be made from extensible materials such as those described in U.S. Pat. No. 6,552,245 to Roessler et al. which is incorporated herein by reference in a manner that is consistent herewith. The topsheet 142 can also be made from biaxially stretchable materials as described in U.S. Pat. No. 6,969,378 to Vukos et al. which is incorporated herein by reference in a manner that is consistent herewith.

The article 20 can optionally further include a surge management layer which may be located adjacent the absorbent core 144 and attached to various components in the article 20 such as the absorbent core 144 or the topsheet 142 by methods known in the art, such as by using an adhesive. Examples of suitable surge management layers are described in U.S. Pat. Nos. 5,486,166 to Bishop et al.; 5,490,846 to Ellis et al.; and 5,820,973 to Dodge et al., each of which is incorporated herein by reference in a manner that is consistent herewith.

The article 20 can further comprise an absorbent core 144. The absorbent core 144 may have any of a number of shapes. For example, it may have a 2-dimensional or 3-dimensional configuration, and may be rectangular shaped, triangular shaped, oval shaped, race-track shaped, 1-shaped, generally hourglass shaped, T-shaped and the like. It is often suitable for the absorbent core 144 to be narrower in the crotch portion 126 than in the rear 124 or front 122 portion(s). The absorbent core 144 can be attached in an absorbent article, such as to the baffle 40 and/or the topsheet 142 for example, by bonding means known in the art, such as ultrasonic, pressure, adhesive, aperturing, heat, sewing thread or strand, autogenous or self-adhering, hook-and-loop, or any combination thereof.

In some desirable aspects, the absorbent core includes cellulose fiber and/or synthetic fiber, such as meltblown fiber, for example. Thus, in some aspects, a meltblown process can be utilized, such as to form the absorbent core 144 in a coform line. In some aspects, the absorbent core 144 can have a significant amount of stretchability.

The absorbent core 144 can additionally or alternatively include absorbent and/or superabsorbent material. Accordingly, the absorbent core 144 can comprise a quantity of superabsorbent material and optionally fluff contained within a matrix of fibers. In some aspects, the total amount of superabsorbent material in the absorbent core 144 can be at least about 10% by weight of the core, such as at least about 30%, or at least about 60% by weight or at least about 90%, or between about 10% and about 98% by weight of the core, or between about 30% to about 90% by weight of the core to provide improved benefits. Optionally, the amount of superabsorbent material can be at least about 95% by weight of the core, such as up to 100% by weight of the core. In other aspects, the amount of absorbent fiber of the present disclosure in the absorbent core 144 can be at least about 5% by weight of the core, such as at least about 30%, or at least about 50% by weight of the core, or between about 5% and 90%, such as between about 10% and 70% or between 10% and 50% by weight of the core. In still other aspects, the absorbent core 144 can optionally comprise about 35% or less by weight unmodified fluff, such as about 20% or less, or 10% or less by weight unmodified fluff.

It should be understood that the absorbent core 144 is not restricted to use with superabsorbent material and optionally fluff. In some aspects, the absorbent core 144 may additionally include materials such as surfactants, ion exchange resin particles, moisturizers, emollients, perfumes, fluid modifiers, odor control additives, and the like, and combinations thereof. In addition, the absorbent core 144 can include a foam.

In one aspect of the disclosure, the color change indicator of the present disclosure may be an ink that is printed or otherwise applied (e.g. by spraying or dipping) to one or more parts of the absorbent article, such as the absorbent materials, either surface of the bodyside liner, either surface of the baffle layer, wings, containment flaps and any other absorbent article component facing the body. In another aspect of the disclosure, the color change indicator may be an integrated component of one or more parts of the absorbent article. For example, pH indicating fibers (fibers having a pH indicator dye immobilized thereon) may make up part of the absorbent core or the like, or may be included in the bodyside liner or baffle material.

The color change indicator changes color when exposed to (a) humidity or moisture for a certain period of time, or (b) placed in contact with a certain quantity of moisture. Higher temperatures can hasten color change due to the fact that there is likely more moisture as a result of the higher temperature and because it causes the pH indicator to react more quickly.

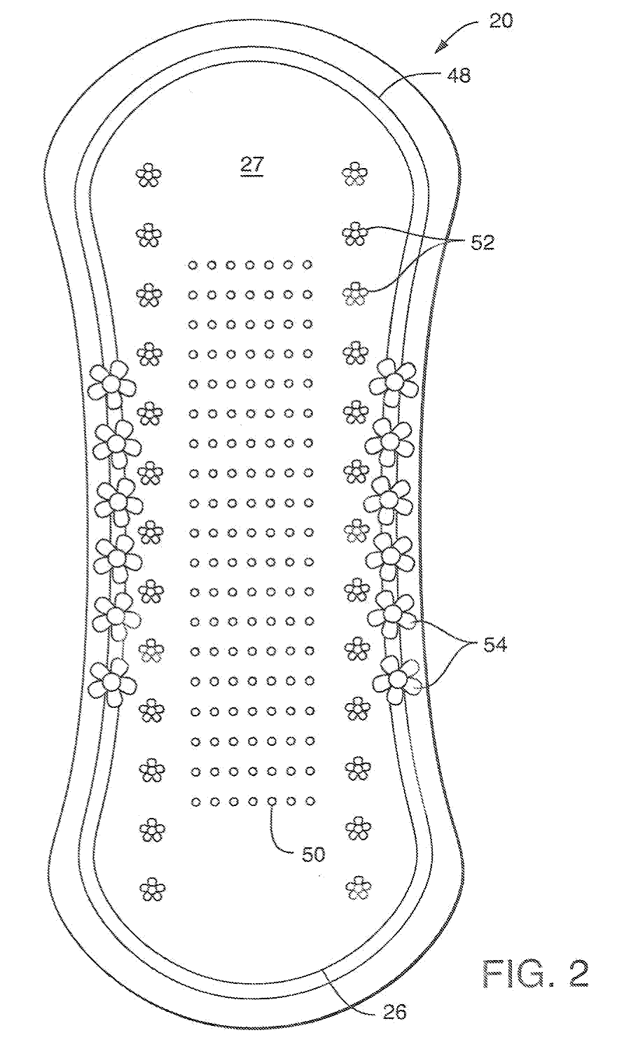

One aspect of the present disclosure is an absorbent article that uses a color change indicator to deliver a signal to a user indicating when the absorbent article contains an undesirable amount of moisture over an undesirable period of time. The color-change indicator changes from an initial color to an alerting color when the product is not appropriate for further use, thereby mitigating the user's risk of being exposed to unhealthy conditions. For example, in the various articles 20 shown in FIGS. 2-4, a color-change indicator is disposed on the article as indicia (e.g. the floral pattern as shown or any other pattern, symbol or alphanumeric character). The color-change indicator is deposited on a select region or component of an absorbent article as described herein. It is further contemplated the color change indicator may be initially colorless but change to a color to provide a signal, or in the alternative, be initially colored but change to colorless to provide the signal.

Generally, the color-change indicator includes at least one base component, at least one acid component and at least one pH indicator composition, all deposited on and/or within a substrate. The acid and base components are physically separated before direct contact with humidity or moisture. Two classes of substrates are described, a matrix substrate and a temporary barrier layer. Each such substrate may coincide with the structure of an absorbent article. For example, a substrate may also operate as a baffle.

In one aspect of the disclosure the substrate is a matrix material. The acid and/or base components may be disposed thereon in solid form. The matrix material allows a controlled release of the acid and/or base components in response to a combination of temperature, moisture and exposure time. Suitable matrix materials include substrates such as cellulosic tissues and papers; nonwovens such as meltblown, coform, spunbond, spunbond-meltblown-spunbond (SMS), bonded-carded-web (BCW), woven fabric; perforated or breathable films; foam and other substrates listed infra.

In another aspect, the acid and/or base may disposed on a surface of the matrix material as a dusting of powder. The powder may be held mechanically within the fibers of the substrate, or adhere to the substrate with an adhesive.

In another aspect, the acid and/or base may be in the form of particles encapsulated in an aqueous-soluble shell material and disposed onto the matrix. These encapsulated particulates may be held mechanically within the fibers of the substrate, or adhere to the substrate with an adhesive. The shell material used for encapsulation may be suitably constructed of a material such that it will release the encapsulated material upon contact with moisture or liquid. The moisture or liquid can cause the shell material to solubilize, disperse, swell, or disintegrate, or the shell material may be permeable such that it disintegrates or discharges the encapsulated material upon contact with the moisture or liquid. Suitable shell materials include cellulose-based polymeric materials (e.g., ethyl cellulose), carbohydrate-based materials (e.g., starches and sugars) and materials derived therefrom (e.g., dextrins and cyclodextrins) as well as other materials compatible with human tissues.

The shell thickness may vary depending upon the material encapsulated, and is generally manufactured to allow the encapsulated component to be covered by a thin layer of encapsulation material, which may be a monolayer or thicker laminate, or may be a composite layer. The layer should be thick enough to resist cracking or breaking of the shell during handling or shipping of the product or during wear which would result in breakage of the encapsulating material.

In yet another aspect, the acid and/or base components may also be disposed within the matrix material in molecular form. In one particular aspect, the matrix material is soaked in an acid or base solution and allowed to dry.

In a further aspect, the acid or base is a powder or particulate embedded in a binding matrix such as a water insoluble ink binder. The ink binder has the characteristic of being able to evaporate quickly so as to avoid putting the acid or base into solution. The resulting ink is then printed onto a substrate. The acid and base may be printed onto the substrate as separate inks.

In another aspect, the matrix is an extruded film that includes particulates and other forms of materials which can be added to the film and which will not chemically interfere with or adversely affect the extruded film, but which are able to be uniformly dispersed throughout the film. Fillers include particulate inorganic materials such as for example talc, calcium carbonate, barium carbonate, magnesium carbonate, magnesium sulfate, titanium dioxide, mica, clays, kaolin, diatomaceous earth and the like, and organic particulate materials such as powdered polymers for example TEFLON and KEVLAR, and wood and other cellulose powders. Some of these fillers are also basic, such as calcium carbonate, negating the need for a separate base material. An ink with an acid component may be applied to a surface of such a matrix material.

In one particular aspect, the matrix is a baffle film prepared by blending an organic or inorganic incompatible filler with a polyolefin-based resin, which is then melted, film-formed and stretched. As indicated herein, such films are mainly used as liquid barriers in disposable personal care products. An ink with an acid component, e.g. citric acid is printed onto either side of the baffle, covering it completely or by applying it to the baffle as indicia or the like as shown in FIG. 3. Desirably, the baffle film is translucent in instances where the color change indicator is applied to the body-facing surface of the baffle.

In yet another aspect of the disclosure, the substrate is a temporary barrier material/layer. The temporary barrier layer may be optically transparent or translucent. Suitable materials for the temporary barrier layer include water soluble substrates, water permeable substrates and water permeable/soluble substrates. In one aspect, the temporary barrier layer is a water-soluble polymer film.

Prior to use, the pH indicator is either in direct contact with the acid or the base, but not both simultaneously. In one aspect, the acid and base are physically deposited on the opposite sides of the temporary barrier layer and are physically separated from each other for a relatively long period of time as the indicator is exposed to a combination of low humidity and low temperature. As an effective amount of moisture collects at the color change indicator, either the acid, the base or a combination thereof will migrate through the temporary barrier layer to neutralize each other to trigger a pH change. The pH change in turn triggers a color change for the user to observe.

The contact between the base and acid is triggered after a moderate amount of exposure time if the indicator is exposed to a combination of high temperature and low moisture level or a combination of low temperature and a high moisture level or a combination of moderate temperature and a moderate moisture level.

The contact between the base and acid is triggered after a short amount of exposure time if the indicator is exposed to a combination of high temperature and high moisture level.

The contact between the base and acid is triggered almost instantaneously (e.g., within 10 minutes) if the indicator is exposed to liquid body fluids such as liquid sweat. A gush of liquid may occur as the result of an insult or high amounts of sweat.

In operation, an absorbent article is donned and its color-change indicator exposed to a combination of low humidity and low temperature. Initially, the base and acid are physically separated from each other for some time, resulting in no color change. Eventually, normal body perspiration and likely a temperature increase will cause the acid and base to neutralize each other and a color change to occur in the pH indicator. This may be expected to happen anywhere from about 5 to 8 hours.

In another example, the reaction between the acid and the base is triggered after a moderate amount of exposure time (e.g., 3 to 5 hours) if the indicator is exposed to a combination of high temperature and low humidity or a combination of low temperature and high humidity or a combination of moderate temperature and moderate humidity.

In another example, the reaction between the acid and the base is triggered after a short amount of exposure time (e.g., 1 to 3 hours) if the indicator is exposed to a combination of high temperature and high humidity.

In another example, the reaction between the acid and the base is triggered almost instantaneously (e.g., within 10 minutes) if the indicator is exposed to liquid body fluids such as liquid sweat.

It is contemplated that more than one pH indicator may be used so as to cover a broader pH range of detection and/or to change the resulting color of the pH indicator.

In a further aspect, the color change indicator may be disposed on any layer of the absorbent article 20 desired. The color-change indicator is typically applied to a layer of the article where it can be easily seen by the user. In FIG. 1, for instance, the color-change indicator as shown may be applied to a body-facing surface 205 of the baffle 28, such as adjacent to a nonwoven web or film of a composite used to form the baffle. If desired, a transparent or translucent region (e.g., window, film, etc.) of the baffle may be employed to allow the color-change indicator to be readily viewed without removal of the absorbent article from the user and/or without disassembly. In other embodiments, the color-change indicator can be seen through a hole or aperture in the absorbent article for observation.

If desired, a plurality of color-change indicators may also be employed to ensure that the signal is adequately conveyed to the user. For example, in certain embodiments, two color-change indicators may be utilized. In still other embodiments, three color-change indicators may be utilized, while in still other embodiments, greater than three color-change indicators may be utilized. The color-change indicators can form either a straight line, a wave, or a curved line (e.g., parabolic). When multiple color-change indicators are employed, they may optionally be arranged in a pattern (e.g., numbers, letters, graphics, etc.). In addition, they may be made with a variety of different pH indicator compositions so that there are stages of signals that may be observed by a user, each stage indicating how close the absorbent article is to a recommended change.

Referring to FIG. 2, shown is one possible example of a feminine-pad type of absorbent article 20. The article 20 has a race-track shape outlined by an embossed pattern 48 which is visible from the surface 27 of top sheet 26. Also visible are optional apertures 50 that function to allow vaginal discharge to more readily penetrate the top sheet 26 to reach an absorbent core. A floral aesthetic feature 52 is oriented along each side of the article 20 in the longitudinal direction. Such features are not critical to the disclosure and are considered optional. The primary focus of the disclosure is one or more color-change indicators located on the article. In this example, color-change indicators are shown as arrays of indicia 54 located along each side of the article 20 in the longitudinal direction of the pad.

The indicia 54 are disposed on the body-facing surface 27 or on the opposite surface thereof. In the latter case, the top sheet 26 is semi-translucent so the indicia is visible there through. In the alternative, the indicia are disposed on a structure located underneath the top sheet 26. Indicia 54 are shaped like flowers, but it is contemplated that they could be any shape. Further, indicia 54 may be a single shape rather than an array. It is most desirable to locate indicia 54 away from the central portion of the article 20 where vaginal discharge would most readily make contact therewith. The intent is to have the indicia 54 make contact primarily with moisture from sweat rather than a vaginal discharge.

Referring to FIG. 3, shown is one possible example of a diaper/pant type of absorbent article 20. On the front region 124, there is a plurality of indicia 150, which are the color-change indicator(s), in the shape of flowers. It is contemplated that such indicia could be any shape, and may be a single indicia such as a stripe or the like. It is most desirable for the indicia 150 to extend from the front region to the back region (not shown). Indicia 150 are disposed on the inner surface of the baffle, wherein the baffle is semi-translucent. However the color change indicator 150 may be located on the garment-facing side. In this aspect, humidity from the environment is detected. Moisture reaching indicia 150 causes a color change. It may be desirable to locate the color change indicator away from the center of the garment and close to the waist band area so that a direct liquid insult does not immediately cause a color change.

Referring now to FIG. 4 is another possible example of a diaper/pant type of absorbent article 20. Like the feminine pad shown in FIG. 2, there is an array of color-change indicators, in the form of indicia 150, located at the garment-facing surface of topsheet 142. Specifically, the indicia 150 may be located on the containment flaps 146, either on the body-facing surface or opposite surface similar to the feminine pad example, supra.

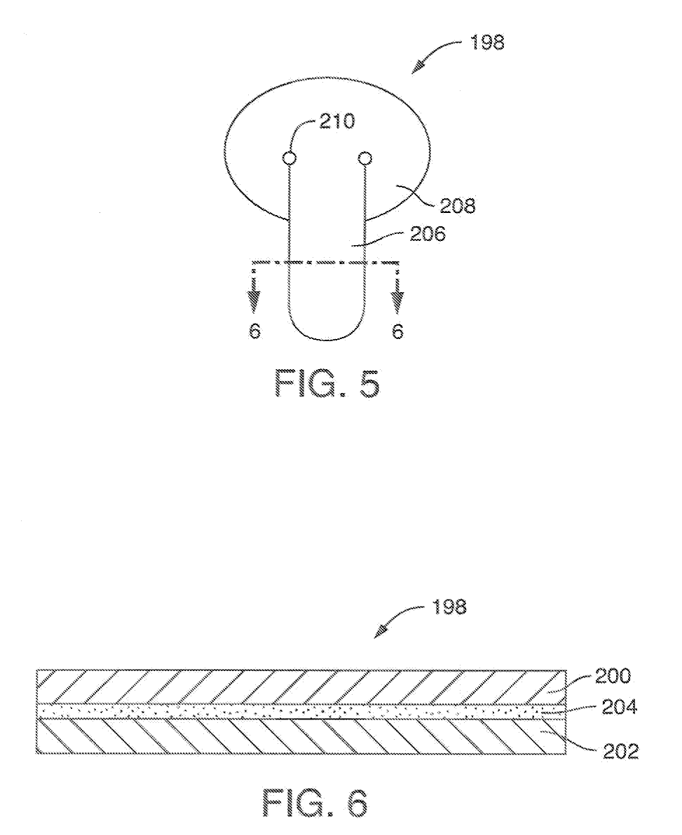

In a further aspect of the disclosure is a color-change indicator in either a stick or sheet form. A color-change indicator sheet may be laminated onto one or more areas of an absorbent article (not shown), such as the body-facing surface of the topsheet 142 or the garment-facing surface of a baffle. A color-change indicator stick sensor, shown as reference 198 in FIG. 5, may be combined with the absorbent article 20 either during manufacture or by a user. For example, as seen in FIG. 3, a user may attach the stick sensor 198 to the waistband of an absorbent article to monitor the environment between the user's body and the absorbent article.

Referring to FIG. 6, in on aspect, the stick sensor 198 (or sheet) may be a laminate made with a nonwoven material 200 to which a color-change indicator solution is applied by spraying, dipping or printing; and a nylon-transfer membrane 202 to which a water solution containing sodium bromocresol green (10 mg/ml), citric acid (10 mg/ml) and has been applied by spraying, dipping or printing. The nonwoven material 200 and nylon-transfer membrane 202 are laminated together using an adhesive 204 to form a sheet. The sheet is cut to create a moisture/temperature/exposure time sensor stick. The stiffness of stick sensor 198 is influenced by the nonwoven materials, the nylon membrane and adhesive used.

In one aspect, the stick sensor 198 is stamped from the laminate sheet shown in FIG. 6 and has features to allow one to clip the stick sensor 198 to a diaper waistband. For example, the stick sensor 198 includes an elongated body 206 having a pair of symmetrically shaped arms 208 extending from one end. As seen in FIG. 3, the arms 208 hold the elongated body 206 in place by functioning as a clip feature. Desirably, to prevent having a stress concentration at each juncture 210 between the arm 208 and elongated body 206, the juncture 210 is defined by a radius.

In another aspect, the stick sensor 198 may be of any shape. For example, the stick sensor 198 may be round, oval, rectangular or any other geometric or fanciful shape such as a flower, star and the like.

In yet another aspect, the stick sensor 198 may forego the arms 208 and instead include a bonding device attached to the nonwoven 200 carrier substrate (not shown). This will create a badge that may be selectively placed on the absorbent article, preferably away from an area of direct insult. The bonding device may be a layer of pressure-sensitive adhesive or clips, pins, snaps, hook material, reticulated foams and the like. Release paper may cover an adhesive layer for protection until use. It is contemplated that badges may be included in packages of absorbent articles for a low cost means to include a color change indicator with an absorbent article.

Suitable examples of pressure-sensitive adhesives include, for instance, acrylic-based adhesives and elastomeric adhesives. In one embodiment, the pressure-sensitive adhesive is based on copolymers of acrylic acid esters (e.g., 2-ethyl hexyl acrylate) with polar co-monomers (e.g., acrylic acid). The adhesive may have a thickness in the range of from about 0.1 to about 2 mils (2.5 to 50 microns).

Examples of substrates used to support a color change indicator and/or make the absorbent articles of the present disclosure include but are not limited to nonwoven webs, woven fabrics, knit fabrics, paper webs, films, foams, strands, etc. Nonwoven webs may include, but are not limited to, spunbonded webs (apertured or non-apertured), meltblown webs, bonded carded webs, air-laid webs, coform webs, hydraulically entangled webs, and so forth. Nonwoven composites (e.g., nonwoven web laminated to a film or strands) may also be employed. Examples of polymers for forming such webs may include, but are not limited to, synthetic polymers (e.g., polyethylene, polypropylene, polyethylene terephthalate, nylon 6, nylon 66, KEVLAR®), syndiotactic polystyrene, liquid crystalline polyesters, etc.); cellulosic polymers (softwood pulp, hardwood pulp, thermomechanical pulp, etc.); combinations thereof; and so forth. Other suitable substrates are mentioned supra.

Suitable acids include organic and inorganic acids. Organic acids may be more desirable and include small molecular acids such as citric acid, oxalic acid, maleic acid, salicylate acid, fatty acids, and macromolecular acids such as polyacrylic acids and polymethacrylic acids. Suitable bases include organic and inorganic bases. Organic bases may be more desirable and include small molecular bases such as sodium bicarbonate, sodium carbonate, sodium hydroxide, sodium borate, amines, and macromolecular bases such as polyamines. The acids and bases may be in powder or particulate form.

As described herein, the pH indicator composition serves to indicate the presence of a bodily fluid such as sweat, urine, etc. The absorbent article of the present invention employs a pH indicator composition that is able to signal to the user to suggest that the article be removed or replaced. A pH adjusted polyamine may serve as an immobilizer of a pH-sensitive composition as well as a buffer, so that the dyes will not leach from the substrate to which it is applied.

The pH-sensitive composition undergoes a change in color at a pH level which occurs when the acid and base react. For example, the pH level at which the color transition occurs may be from about 3.5 to 9.5, in some embodiments from about 4.5 to 8.5 and in some embodiments, from about 5 to 6.5, and in one embodiment, about 5. The pH-sensitive composition may, for instance, exhibit a first color at pH values of less than about 9, in some embodiments less than about 8, in some embodiments less than about 7, in some embodiments less than about 6, in some embodiments less than about 5, and in some embodiments, less than about 4. Likewise, the pH-sensitive composition may also exhibit a second color at pH values of about 4 or more, in some embodiments about 5 or more, in some embodiments about 6 or more, in some embodiments about 7 or more, in some embodiments about 8 or more, and in some embodiments, about 9 or more. The first color may be present when the composition is in its dry state, and the second color may be present when the composition comes into contact with the bodily fluid. It should be noted that the term “color” as used herein includes a composition that is generally clear or colorless.

The pH-sensitive composition employs one or more chromogens to achieve the desired color change. The particular chromogens employed in the pH-sensitive composition are not generally critical. For instance, phthalein chromogens constitute one class of suitable pH-sensitive chromogens that may be employed in the present disclosure. Phenol Red (i.e., phenolsulfonephthalein), for example, exhibits a transition from yellow to red over the pH range 6.6 to 8.0. Above a pH of about 8.1, Phenol Red turns a bright pink (fuchsia) color. Derivatives of Phenol Red can also be suitable for use in the present disclosure, such as those substituted with chloro, bromo, methyl, sodium carboxylate, carboxylic acid, hydroxyl and amine functional groups. Exemplary substituted Phenol Red compounds include, for instance, Metacresol Purple (meta-cresolsulfonephthalein), Cresol Red (ortho-cresolsulfonephthalein), Pyrocatecol Violet (pyrocatecolsulfonephthalein), Chlorophenol Red (3′,3″-dichlorophenolsulfonephthalein), Xylenol Blue (the sodium salt of para-xylenolsulfonephthalein), Xylenol Orange, Mordant Blue 3 (C.I. 43820), 3,4,5,6-tetrabromophenolsulfonephthalein, Bromoxylenol Blue, Bromophenol Blue (3′,3″,5′,5″-tetrabromophenolsulfonephthalein), Bromochlorophenol Blue (the sodium salt of dibromo-5′,5″-dichlorophenolsulfonephthalein), Bromocresol Purple (5′,5″-dibromo-ortho-cresolsulfonephthalein), Bromocresol Green (3′,3″,5′,5″-tetrabromo-ortho-cresolsulfonephthalein), and so forth. For example, Bromocresol Green exhibits a transition from yellow to blue over a pH range of about 4 to about 6; Bromothymol Blue exhibits a transition from yellow to blue over a pH range of about 6.0 to 7.6; Bromophenol Blue exhibits a transition from yellow to violet over a pH range of about 3.0 to 4.6; and Bromocresol Purple exhibits a transition from yellow to violet over a pH of about 5.2 to 6.8.

Anthraquinones constitute another suitable class of pH-sensitive chromogens for use in the present disclosure. Anthraquinones have the following general structure:

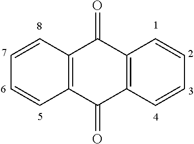 [SMILES: O=C1c2ccccc2C(=O)c2ccccc21]

The numbers 1-8 shown in the general formula represent a location on the fused ring structure at which substitution of a functional group can occur. Some examples of such functional groups that may be substituted on the fused ring structure include halogen groups (e.g., chlorine or bromine groups), sulfonyl groups (e.g., sulfonic acid salts), alkyl groups, benzyl groups, amino groups (e.g., primary, secondary, tertiary, or quaternary amines), carboxy groups, cyano groups, hydroxy groups, phosphorous groups, etc. Functional groups that result in an ionizing capability are often referred to as “chromophores.” Substitution of the ring structure with a chromophore causes a shift in the absorbance wavelength of the compound. Thus, depending on the type of chromophore (e.g., hydroxyl, carboxyl, amino, etc.) and the extent of substitution, a wide variety of quinones may be formed with varying colors and intensities. Other functional groups, such as sulfonic acids, can also be used to render certain types of compounds (e.g., higher molecular weight anthraquinones) water-soluble.

Some suitable anthraquinones that may be used in the present disclosure, as classified by their “Cl” number, include Acid Black 48, Acid Blue 25 (D&C Green No. 5), Acid Blue 40, Acid Blue 41, Acid Blue 45, Acid Blue 80, Acid Blue 129, Acid Green 25, Acid Green 27, Acid Green 41, Acid Violet 43, Mordant Red 11 (Alizarin), Mordant Black 13 (Alizarin Blue Black B), Mordant Red 3 (Alizarin Red S), Mordant Violet 5 (Alizarin Violet 3R), Alizarin Complexone, Natural Red 4 (Carminic Acid), Disperse Blue 1, Disperse Blue 3, Disperse Blue 14, Natural Red 16 (Purpurin), Natural Red 8, Reactive Blue 2 (Procion Blue HB), Reactive Blue 19 (Remazol Brilliant Blue R); Alizarin, Alizarin Yellow R, Alizarin Yellow GG, Alizarin S, Nuclear Fast Red, Quinalizarin, Emodin, amino-4-hydroxyanthraquinone, and so forth. For instance, carminic acid exhibits a first transition from orange to red over a pH range of about 3.0 to 5.5 and a second transition from red to purple over a pH range of about 5.5 to 7.0.

Yet another suitable class of pH-sensitive chromogens that may be employed is aromatic azo compounds having the general structure:

X—R₁—N═N—R₂—Y

wherein,
R₁ is an aromatic group;
R₂ is selected from the group consisting of aliphatic and aromatic groups; and
X and Y are independently selected from the group consisting of hydrogen, halides, —NO₂, —NH₂, aryl groups, alkyl groups, alkoxy groups, sulfonate groups, —SO₃H, —OH, —COH, —COOH, halides, etc. Also suitable are azo derivatives, such as azoxy compounds (X—R₁—N═NO—R₂—Y) or hydrazo compounds (X—R₁—NH—NH—R₂—Y). Particular examples of such azo compounds (or derivatives thereof) include Methyl Violet, Methyl Yellow, Methyl Orange, Methyl Red, and Methyl Green. For instance, Methyl Yellow undergoes a transition from red to yellow at a pH range of about 2.9 to 4.0, Methyl Orange undergoes a transition from red to yellow at a pH range of about 3.1 to 4.4, and Methyl Red undergoes a transition from red to yellow at a pH range of about 4.2 to 6.3.

Arylmethanes (e.g., diarylmethanes and triarylmethanes) constitute still another class of suitable pH-sensitive chromogens for use in the present disclosure.

Triarylmethane leuco bases, for example, have the following general structure:

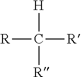

wherein R, R′, and R″ are independently selected from substituted and unsubstituted aryl groups, such as phenyl, naphthyl, anthracenyl, etc. The aryl groups may be substituted with functional groups, such as amino, hydroxyl, carbonyl, carboxyl, sulfonic, alkyl, and/or other known functional groups. Examples of such triarylmethane leuco bases include Leucomalachite Green, Pararosaniline Base, Crystal Violet Lactone, Crystal Violet Leuco, Crystal Violet, CI Basic Violet 1, CI Basic Violet 2, CI Basic Blue, CI Victoria Blue, N-benzoyl leuco-methylene, etc. Likewise suitable diarylmethane leuco bases can include 4,4′-bis(dimethylamino)benzhydrol (also known as “Michler's hydrol”), Michler's hydrol leucobenzotriazole, Michler's hydrol leucomorpholine, Michler's hydrol leucobenzenesulfonamide, etc.

Still other suitable pH-sensitive chromogens that may be employed include Congo Red, Litmus (azolitmin), Methylene Blue, Neutral Red, Acid Fuchsin, Indigo Carmine, Brilliant Green, Picric acid, Metanil Yellow, m-Cresol Purple, Quinaldine Red, Tropaeolin O, 2,6-dinitrophenol, Phloxine B, 2,4-dinitrophenol, 4-dimethylaminoazobenzene, 2,5-dinitrophenol, 1-Naphthyl Red, Chlorophenol Red, Hematoxylin, 4-nitrophenol, nitrazine yellow, 3-nitrophenol, Alkali Blue, Epsilon Blue, Nile Blue A, universal chromogens, and so forth. For instance, Congo Red undergoes a transition from blue to red at a pH range of about 3.0 to 5.2 and Litmus undergoes a transition from red to blue at a pH range of about 4.5 to 8.3.

Although the overall amount may vary, the pH chromogen(s) typically constitute from about 0.01 wt. % to about 15 wt. %, in some embodiments from about 0.1 wt. % to about 5 wt. %, and in some embodiments, from about 0.2 wt. % to about 1 wt. %, of the pH-sensitive composition on a dry basis.

Of course, the pH-sensitive composition may also contain a variety of optional components to facilitate the desired color change, and also to enhance the ability of the composition to remain stable on a substrate of the feminine care absorbent article to which it is applied. Organic binders may, for instance, be employed to increase the durability of the pH-sensitive composition and help form stable films on various substrates upon drying. Because the composition is intended for contact with aqueous bodily fluids (e.g., urine), it is sometimes desired that hydrophobic organic binders are employed. One example of such a binder is a thermoset resin that is capable of hardening upon application to the substrate. Suitable thermoset resins may include, for instance, polyester resins, polyurethane resins, melamine resins, epoxy resins, diallyl phthalate resins, vinylester resins, and so forth. In addition or in conjunction with such hydrophobic binders, the composition may also contain a hydrophilic binder, such as alginic acid and salts thereof, carrageenan, pectin, gelatin and the like, semisynthetic macromolecular compounds, such as methylcellulose, cationized starch, carboxymethylcellulose, carboxymethylated starch, nitrocellulose, vinyl polymers (e.g., polyvinyl alcohol), polyvinylpyrrolidone, polyacrylic acid, polyacrylamide, maleic acid copolymers, cellulose acetate, cellulose butyrate, etc., as well as combination thereof. Commercially available binder systems that may be employed include, for instance, the GANTREZ® SP, ES, or AN series of monoalkyl esters of poly(methyl vinyl ether/maleic acid) (International Specialty Products, Inc.), the DERMACRYL® series of carboxylated acrylic copolymers (Akzo Nobel), and the AMPHOMER® series of amphoteric acrylic copolymers (Akzo Nobel).

The total concentration of binders may generally vary depending on the desired properties of the substrate at which the color change indicator is applied. For instance, high total binder concentrations may provide better physical properties for the coated substrate, but may likewise have an adverse effect on other properties, such as the absorptive capacity of the substrate to which it is applied. Conversely, low total binder concentrations may not provide the desired degree of durability. In most embodiments, however, the total amount of binder employed in the composition, including any hydrophilic or hydrophobic binders, is from about 20 wt. % to about 90 wt. %, in some embodiments from about 40 wt. % to about 85 wt. %, and in some embodiments, from about 60 wt. % to about 80 wt. %, on a dry weight basis.

The pH-sensitive composition may also contain other components as is known in the art. For example, a wetting agent may sometimes be employed to improve the ability to apply and adhere the pH-sensitive composition to a substrate. Suitable wetting agents may include, for instance, a surfactant (e.g., nonionic, cationic, anionic, or zwitterionic) or a mixture of surfactants. The surfactants may also help enhance the sensitivity and contrast provided by the colorant. Particularly desired surfactants are nonionic surfactants, such as ethoxylated alkylphenols, ethoxylated and propoxylated fatty alcohols, ethylene oxide-propylene oxide block copolymers, ethoxylated esters of fatty (C8-C18) acids, condensation products of ethylene oxide with long chain amines or amides, condensation products of ethylene oxide with alcohols, acetylenic diols, and mixtures thereof. Various specific examples of suitable nonionic surfactants include, but are not limited to, methyl gluceth-10, PEG-20 methyl glucose distearate, PEG-20 methyl glucose sesquistearate, C11-C15 pareth-20, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C6-C22) alcohol, including 3 to 20 ethylene oxide moieties, polyoxyethylene-20 isohexadecyl ether, polyoxyethylene-23 glycerol laurate, polyoxy-ethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxy-ethylene-6 tridecyl ether, laureth-2, laureth-3, laureth-4, PEG-3 castor oil, PEG 600 dioleate, PEG 400 dioleate, and mixtures thereof. Commercially available nonionic surfactants may include the SURFYNOL® range of acetylenic diol surfactants available from Air Products and Chemicals of Allentown, Pa. and the TWEEN® range of polyoxyethylene surfactants available from Fischer Scientific of Pittsburgh, Pa. Cationic surfactants may also be employed in the present invention, such as quaternary ammonium compounds (e.g., cetyl trimethyl ammonium chloride, benzalkonium chloride, benzethonium chloride, quaternium-18, stearalkonium chloride, cocotrimonium methosulfate, PEG-2 cocomonium chloride, and PEG-3 dioleoylamidoethylmonium methosulfate, etc). In certain embodiments, such cationic surfactants may also aid in adhering the composition to a substrate having a negatively charged surface, such as films and/or nonwoven webs formed from olefinic polymers. When employed, such wetting agents typically constitute from about 0.01 Wt. % to about 20 wt. %, in some embodiments from about 0.1 wt. % to about an about 15 wt. %, and in some embodiments, from about 1 wt. % to about 10 wt. % of the composition.

The initial pH of the composition may also be controlled within a certain range to ensure that it exhibits a first color before use of the article, and then undergoes a color change upon contact with liquid sweat. The pH may also be such that humidity from the environment alone (e.g., in storage or use) does not induce the color change. For instance, it is typically desired that the initial pH of the composition is within a range of from about 3 to about 6, and in some embodiments, from about 4 to about 6. Various pH modifiers may be employed to achieve the desired pH level. Some examples of pH modifiers that may be used in the present invention include, but are not limited to, mineral acids, sulfonic acids (e.g., 2-[N-morpholino]ethane sulfonic acid), carboxylic acids, and polymeric acids. Specific examples of suitable mineral acids are hydrochloric acid, nitric acid, phosphoric acid, and sulfuric acid. Specific examples of suitable carboxylic acids are lactic acid, acetic acid, citric acid, glycolic acid, maleic acid, gallic acid, malic acid, succinic acid, glutaric acid, benzoic acid, malonic acid, salicylic acid, gluconic acid, and mixtures thereof. Specific examples of suitable polymeric acids include straight-chain poly(acrylic) acid and its copolymers (e.g., maleic-acrylic, sulfonic-acrylic, and styrene-acrylic copolymers), cross-linked polyacrylic acids having a molecular weight of less than about 250,000, poly(methacrylic) acid, and naturally occurring polymeric acids such as carageenic acid, carboxymethyl cellulose, and alginic acid. While the amount of pH modifiers will generally depend on the desired pH level, such components typically constitute from about 1 wt. % to about 40 wt. %, in some embodiments from about 5 wt. % to about 30 wt. %, and in some embodiments, from about 10 wt. % to about 25 wt. % of the composition.

Humectants may also be utilized, such as ethylene glycol; diethylene glycol; glycerine; polyethylene glycol 200, 300, 400, and 600; propane 1,3 diol; propylene-glycolmonomethyl ethers, such as Dowanol PM (Gallade Chemical Inc., Santa Ana, Calif.); polyhydric alcohols; or combinations thereof. Various other components may also be employed, such as colorant stabilizers, photoinitiators, fillers, etc., such as described in U.S. Pat. Nos. 5,681,380 to Nohr, et al. and 6,542,379 to Nohr, et al., which are incorporated herein in their entirety by reference thereto for all purposes. Typically, the components of the pH indicator composition are initially dissolved or dispersed in a solvent to form a coating solution. Any solvent capable of dispersing or dissolving the components is suitable. Suitable solvents may include, for instance, water; alcohols, such as ethanol or methanol; dimethylformamide; dimethyl sulfoxide; hydrocarbons, such as pentane, butane, heptane, hexane, toluene and xylene; ethers such as diethyl ether and tetrahydrofuran; ketones and aldehydes, such as acetone and methyl ethyl ketone; halogenated solvents, such as dichloromethane and carbon tetrachloride; acrylonitrile; etc., as well as mixtures thereof. The concentration of solvent in the coating formulation is generally high enough to allow easy application, handling, etc.

When employed, the total concentration of solvent(s) may vary, but is typically from about 1 wt. % to about 95 wt. %, in some embodiments from about 5 wt. % to about 80 wt. %, and in some embodiments, from about 10 wt. % to about 50 wt. % of the coating formulation. A coating formulation may be applied using any conventional technique, such as printing, dipping, spraying, melt extruding, coating (e.g., solvent coating, powder coating, brush coating, etc.), and so forth.

In one embodiment, for example, the pH-sensitive composition and one other component of the color change indicator is printed onto one substrate (e.g., baffle). A variety of printing techniques may be used for applying the composition to the support, such as gravure printing, flexographic printing, screen printing; laser printing, thermal ribbon printing, piston printing, etc. In one particular embodiment, ink-jet printing techniques are employed to apply the composition to the substrate. Ink-jet printing is a non-contact printing technique that involves forcing an ink through a tiny nozzle (or a series of nozzles) to form droplets that are directed toward the support. Two techniques are generally utilized, i.e., “DOD” (Drop-On-Demand) or “continuous” ink-jet printing. In continuous systems, ink is emitted in a continuous stream under pressure through at least one orifice or nozzle. The stream is perturbed by a pressurization actuator to break the stream into droplets at a fixed distance from the orifice. DOD systems, on the other hand, use a pressurization actuator at each orifice to break the ink into droplets. The pressurization actuator in each system may be a piezoelectric crystal, an acoustic device, a thermal device, etc. The selection of the type of ink jet system varies on the type of material to be printed from the print head. For example, conductive materials are sometimes required for continuous systems because the droplets are deflected electrostatically. Thus, when the sample channel is formed from a dielectric material, DOD printing techniques may be more desirable.

A coating formulation may be applied to one or both surfaces of the substrate. For example, the resulting pH-sensitive composition is generally present on at least the surface of the substrate that is likely to contact bodily fluids during use. When applying the composition to multiple surfaces, each surface may be coated sequentially or simultaneously. Regardless of the manner in which it is applied, the resulting substrate may be dried at a certain temperature to drive the solvent from the formulation and form the composition of the present invention. For example, the substrate may be dried at a temperature of at least about 20° C., in some embodiments at least about 25° C., and in some embodiments, from about 25° C. to about 75° C.

To maintain absorbency, porosity, flexibility, and/or some other characteristic of the substrate, it may sometimes be desired to apply the color change composition so as to cover less than 100%, in some embodiments from about 10% to about 80%, and in some embodiments, from about 20% to about 60% of the area of one or more surfaces of the substrate. For instance, in one particular embodiment, the composition is applied to a substrate in a preselected pattern (e.g., reticular pattern, diamond-shaped grid, dots, and so forth). The composition may also be applied uniformly to one or more surfaces of the substrate. In addition, a patterned composition may also provide different functionality to each zone. For example, in one embodiment, the substrate is treated with two or more patterns of coated regions that may or may not overlap. The regions may be on the same or different surfaces of the substrate. In one embodiment, one region of a substrate is coated with a first composition, while another region is coated with a second composition.

It is noted that response time for a color change occur is dependent on the substrate used: the response time is slower when the substrate is less breathable resulting from less encapsulated weak base. Response time also depends on the amount of ink used: the response time is slower with more ink. The response time further depends on the ink formulations as well as the percentage of each ink component. Overall, given these factors, the response time can be easily tailored for different applications.

It is generally desired that a pH-sensitive composition is applied in a manner so that it does not substantially diffuse through the substrate (i.e., non-diffusively immobilized). This enables a user to readily detect the change in color that occurs and also prevents the composition from leaching out of the substrate. The immobilization may be achieved by many methods such as chemical bonding (ionic bonding, covalent bonding, etc.), physical absorption, or using a carrier. In one embodiment, for example, a cationic material (e.g., cationic surfactant) is employed to help ionically adhere the composition to a negatively charged substrate material. In other embodiments, an anchoring compound may be employed that links the ink to the surface of substrate and further improves durability. Typically, the anchoring compound is larger in size than the chromogen, which improves their likelihood of remaining on the surface during use. For example, the anchoring compound can include a macromolecular compound, such as a polymer, oligomer, dendrimer, particle, etc. Polymeric anchoring compounds can be natural, synthetic, or combinations thereof. Examples of natural polymeric anchoring compounds include, for instance, polypeptides, proteins, DNA/RNA and polysaccharides (e.g., glucose-based polymers, activated dextran, etc). In some embodiments, the anchoring compound can be a particle (sometimes referred to as a “bead” or “microbead”). Naturally occurring particles, such as nuclei, mycoplasma, plasmids, plastids, mammalian cells (e.g., erythrocyte ghosts), unicellular microorganisms (e.g., bacteria), polysaccharides (e.g., agarose), etc., can be used. Further, synthetic particles can also be utilized. For example, in one embodiment, latex microparticles are utilized. Although any synthetic particle can be used, the particles are typically formed from polystyrene, butadiene styrenes, styreneacrylic-vinyl terpolymer, polymethylmethacrylate, polyethyl methacrylate, styrene-maleic anhydride copolymer, polyvinyl acetate, polyvinylpyridine, polydivinylbenzene, polybutyleneterephthalate, acrylonitrile, vinylchloride-acrylates, and so forth, or an aldehyde, carboxyl, amino, hydroxyl, or hydrazide derivative thereof. When utilized, the size of the particles may vary. For instance, the average size (e.g., diameter) of the particles can range from about 0.1 nanometers to about 1,000 microns, in some embodiments, from about 0.1 nanometers to about 100 microns, and in some embodiments, from about 1 nanometer to about 10 microns.

Color change may be represented by a certain change in the absorbance reading as measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;
a*=Red/green axis, ranging approximately from −100 to 100; positive values are reddish and negative values are greenish; and
b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed E and calculated by taking the square root of the sum of the squares of the three differences (L*, a*, and b*) between the two colors. In CIELAB color space, each E unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model # CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Typically, the color change is represented by a □E of about 2 or more, in some embodiments about 3 or more, and in some embodiments, from about 5 to about 50.

The color change indicator may also be able to maintain its signal strength (i.e., change in color) for a long enough period of time to ensure that the user is able to detect the change in appearance. For example, the color change indicator may be able to maintain signal strength for at least about 30 minutes, in some embodiments at least about 120 minutes, and in some embodiments, at least about 3 hours. Additionally, the sensor may be subjected to multiple urine insults and still produce accurate test results.

The color change indicator composition may contain a preservative or preservative system to help further inhibit the growth of microorganisms over an extended period of time. Suitable preservatives may include, for instance, alkanols, disodium EDTA (ethylenediamine tetraacetate), EDTA salts, EDTA fatty acid conjugates, isothiazolinone, benzoic esters (parabens) (e.g., methylparaben, propylparaben, butylparaben, ethylparaben, isopropylparaben, isobutylparaben, benzylparaben, sodium methylparaben, and sodium propylparaben), and so forth.

The present invention may be better understood with reference to the following example.

### EXAMPLES

### Example 1

An ink formulation A with bromocresol green and citric acid was printed on the inner side of a baffle layer of a feminine care pad. When the pad is unused or used but not contaminated with a significant quantity of sweat, the ink remains yellow. When liquid sweat is in contact with the color change indicator, the sweat facilitates neutralization reaction between the acid in the ink and calcium carbonate in the baffle layer to cause a rapid color change. The yellow color turns to green/blue. When the indicator becomes green/blue, the pad is discarded.

### Example 2

A moisture/temperature/exposure time sensitive ink containing 1% bromocresol green sodium salt, 8% citric acid, 4% benzethenium chloride, 50% nitrocellulose varnish and 37% ethanol was prepared. The ink was printed on a polyethylene baffle material, made breathable with calcium carbonate, to create a baffle sample. The sample was air dried. When dry, the ink appeared yellow.

One baffle sample was placed in an oven at 37° C., 90% humidity. The yellow turned green after two hours exposure, and then turned to blue after four total hours of exposure. When an identical sample was sealed in a plastic bag prior to being exposed to the noted condition, the yellow hue did not change.

Another baffle sample was used to replace the baffle of two HUGGIES brand new-born diapers. One diaper was placed in an oven at 37° C., 90% humidity. The yellow hue turned green after four hours exposure. When an identical sample was sealed in a plastic bag prior to being exposed to the noted condition, the yellow hue did not change.

### Example 3

Preparation of humidity/temperature/exposure time sensing sticks are as follows. A sample of KIMWIPES brand nonwoven material was soaked in a solution of 1 g sodium carbonate and 20 ml water, and then air dried. A sample of yellow BIODYNE PLUS brand nylon-transfer membrane (available from Pall Corporation, Port Washington, N.Y.) was soaked in a solution of sodium bromocresol green (10 mg/ml), citric acid (10 mg/ml) in water, and then air dried. The KIMWIPES brand nonwoven fabric and the nylon-transfer membrane samples were laminated together using double-sided tape to create a humidity/temperature/exposure time sensor stick. The sensor stick was approximately 5 cm by 5 cm. The sensor stick was taped to the waist band of a HUGGIES brand diaper and placed in an oven at 37° C., 90% humidity. The yellow hue of the membrane turned green after three hours of exposure. When an identical sample was sealed in a plastic bag prior to exposure to the noted condition, the yellow hue did not change.

### Example 4

A piece of highly breathable polypropylene film containing calcium carbonate was printed with an ink containing: 1% bromocresol, 9.4% citric acid, 2.1% polyacrylic acid, 1.5% benzethenium chloride, 35% varnish, and 47.6% ethanol, and air dried. The film was cut into samples. The dried ink had a yellow hue. The yellow hue did not change significantly when a different film samples were exposed to 25% humidity, 35% humidity, 45% humidity at 30° C. respectively, for one week. The yellow hue turned blue on a sample exposed to 95% humidity at 30° C. for five hours.

### Example 5

Samples prepared in the same manner as those in Example 5 remained yellow when exposed to 85% humidity at 15° C., 20° C. and 25° C. for three days. The yellow hue turned blue on a sample exposed to 37° C. for three hours.

### Example 6

Samples prepared in the same manner as those in Example 5 remained yellow when exposed to when exposed to 85% humidity at 40° C. for 10 minutes, 30 minutes and one hour, respectively. The yellow hue turned yellow-green after two hours. The yellow-green hue turned green-blue after 3 hours total exposure time. The green-blue hue turned blue after 10 hours total exposure time.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

1. A personal care absorbent article comprising:
a baffle;
a liquid-permeable topsheet;
an absorbent core positioned between the baffle and the topsheet; and
a color change indicator disposed on the baffle and/or the absorbent core and/or the topsheet;
the color change indicator comprising a pH indicator composition, an acid component and a base component;
wherein the acid component and the base component are physically separated; and
wherein the acid component and the base component are configured to chemically react with one another in the presence of an effective amount of water.

2. The personal care absorbent article of claim 1, wherein the acid component comprises an organic acid.

3. The personal care absorbent article of claim 1, wherein the base component comprises an organic base.

4. The personal care absorbent article of claim 1, wherein the color-change indicator is disposed on a body-facing surface of the baffle.

5. The personal care absorbent article of claim 1, wherein the baffle is a matrix material comprising a basic component.

6. The personal care absorbent article of claim 5, wherein an ink comprising an acid component is disposed on a surface of the baffle.

7. The personal care absorbent article of claim 1, wherein the pH indicator composition is separated from either the acid component or the base component and contacts the remaining acid component or the base component.

8. The personal care absorbent article of claim 1, wherein the topsheet comprises a temporary barrier layer, wherein the acid component is disposed on a surface of the topsheet, and the basic component is disposed on an opposite surface of the topsheet.

9. The personal care absorbent article of claim 1, wherein the acid component and/or the base component are encapsulated.

10. The personal care absorbent article of claim 1, wherein the acid component or the base component is disposed on or within a matrix material.

11. The personal care absorbent article of claim 10 wherein the matrix material is selected from the groups consisting of: cellulosic tissues and papers, nonwovens, woven fabric, perforated or breathable films and foam.

12. The personal care absorbent article of claim 1 wherein the acid component or the base component is disposed within a matrix material, wherein the matrix material comprises an ink binder.

13. The personal care absorbent article of claim 12 wherein the ink binder is water insoluble.

14. The personal care absorbent article of claim 1 further comprising a second color change indicator.

15. A color change indicator comprising:
a pH indicator composition, an acid component and a base component;
wherein the acid component and the base component are physically separated;
wherein the pH indicator composition makes contact with only the acid component or the base component; and
wherein the acid component and the base component are configured to chemically react with one another in the presence of an effective amount of water.

16. The color change indicator of claim 15 further comprising a substrate to which the pH indicator composition, the acid component and the base component are attached.

17. The color change indicator of claim 16 wherein the color change indicator further comprises a bonding device disposed on a surface the substrate.

18. The color change indicator of claim 15 wherein the substrate is in the form of a stick that is configured to be attached to an absorbent garment.

19. The color change indicator of claim 15 wherein the acid component is selected from the group consisting of citric acid, oxalic acid, maleic acid, salicylate acid, fatty acid, polyacrylic acid and polymethacrylic acid.

20. The color change indicator of claim 15 wherein the base component is selected from the group consisting of calcium carbonate, sodium bicarbonate, sodium carbonate, sodium hydroxide, sodium borate, amine and polyamine.
